(19) United States
(12) Patent Application Publication
(10) Pub. No.: US 2008/0082149 A1
(43) Pub. Date: Apr. 3, 2008

(54) Laser treatment of pigmented lesions on the skin

(76) Inventor: Eric F. Bernstein, Gladwyne, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/898,435

(22) Filed: Sep. 12, 2007

Related U.S. Application Data
(60) Provisional application No. 60/844,107, filed on Sep. 13, 2006.

(51) Int. Cl. A61N 5/067

(52) U.S. Cl. 607/89; 128/898; 606/9

(57) ABSTRACT

The present invention relates in general to laser treatment of skin imperfections. The invention specifically relates to treatment of unwanted vasculature and pigmentation with the same device using two or more specific wavelengths of laser light.

This application claims priority to U.S. Provisional Application No. 60/844,107, filed Sep. 13, 2006, which is incorporated herein by reference in its entirety.

The traits associated with skin aging are largely due to chronic sun exposure. Five main changes occur in photodamaged skin: fine lines and wrinkles, enlarged pores, spider veins, sagging skin, and brown spots such as solar lentigos and ephiledes (freckles). The pulsed-dye laser has been the mainstay of removing unwanted blood vessels such as those that occur in port-wine stain birthmarks, in sun-damaged skin, in those with rosacea, and in scars for example since the 1980s. The 577-600 nm laser light typically made available from the pulsed-dye laser is also absorbed by melanin pigment, but the having such strong absorption in hemoglobin competing for this wavelength has made these lasers less than optimal for treating pigmented lesions such as ephiledes and solar lentigos. The pulsed-dye laser has been shown to improve wrinkles in sun-damaged skin presumably due to the inflammation that occurs immediately following treatment.

Today a number of devices are being used to treat unwanted blood vessels in sun-damaged skin. Green light in the 532 nm range is used to treat unwanted blood vessels and targets hemoglobin, and because of it's relatively short wavelength also targets melanin pigment, but suffers the same problems as orange 595 nm laser light in being so strongly absorbed by hemoglobin. This competes with melanin absorption. In addition, 532 nm light is so strongly absorbed by melanin pigment, that damage to the skin occurs more easily than with longer wavelengths, because the light is absorbed well by normal melanin and not preferentially enough by ephiledes and solar lentigos.

Some devices use filtered intense pulsed light that contains many wavelengths of light. The advantage of these systems is that they are inexpensive to produce, and can be used to treat a range of conditions in the skin with less specificity and potentially greater side-effects. Intense pulsed light devices that emit broad spectrum light are not optimal for treating blood vessels since much of the emitted light is not absorbed by hemoglobin and thus contributes to non-specific heating of the skin, and thus side-effects. Melanin, unlike hemoglobin, has a very large range of absorbing wavelengths, and can be targeted by intense pulsed light devices fairly effectively, although over treatment will result in considerable side effects. Melanin absorbs the shorter wavelengths best, since these are the most damaging to our body and melanin has presumably evolved as a protective mechanism against solar radiation. Therefore, ultraviolet wavelengths from 290-400 nm are absorbed the strongest, with decreasing absorption at longer wavelengths, absorbing into the infrared.

The pulsed-dye laser is used primarily in the 585-595 nanometer wavelengths because of its specificity for hemoglobin absorption. This laser targets blood vessels specifically enabling their safe removal with a minimum of side effects. To reduce side effects from heating of the surface epidermis which overlies dermal blood vessels, various cooling devices have been developed that: apply a cold sapphire plate to the surface of the skin, spray cold air on the skin, or spray a cryogen that cools the surface of the skin by evaporation while allowing the unwanted deeper vessels to be damaged by the laser heat hastening their removal. Because hemoglobin absorbs so well at the typical wavelengths used in the pulsed-dye laser, little improvement in freckles or solar lentigos is achieved following pulsed-dye laser treatment.

The addition of epidermal cooling devices to enable the use of higher energies without damaging the skin and decrease the discomfort of treatments has further decreased the beneficial effect of these lasers on unwanted epidermal melanin pigment. A recent simple invention has made the treatment of epidermal pigmented lesions such as ephiledes and solar lentigos much more effective with the pulsed-dye laser. This device simply compresses the skin with a lens that is convex on one side, pushing most of the blood out of the skin underlying the lens. This has the effect of allowing laser light, typically 595 nanometers in wavelength, to enter the skin and have very little hemoglobin absorption, since most of the hemoglobin within the skin has been pushed away by the pressure of the lens. The light can then exit the skin through the epidermis a second time, more effectively treating ephiledes and lentigos. Ephiledes and lentigos more effectively absorb the light as compared to surrounding skin because they contain more melanin pigment.

The drawback of the foregoing method is that individual lesions must be treated one or a few at a time, and often when the entire skin surface is treated during the same session that pigmented spots are treated, purpura (bruising) results at the site of the treated freckles or solar lentigos. Purpura results because the treating wavelength has some absorption by the little hemoblobin present in the compressed skin, and a second treatment is sufficient to produce purpura in these doubly-treated areas.

This invention seeks to make treatment of pigmented lesions with the pulsed-dye laser faster, easier and more efficacious. Pulsed-dye lasers are capable of emitting a variety of wavelengths of light depending upon the dye used and the use of diffraction gradients or prisms to select various wavelengths from the spectrum of light emitted by exciting the laser dye. To make treatment sun-damaged skin or skin containing pigmented spots from other causes, we describe here a dye laser capable of emitting wavelengths away from the peak absorption of hemoglobin for the specific purpose of treating pigmented lesions.

This invention describes the development of a dye laser with tunable wavelengths for the treatment of pigmentation in skin such as solar lentigos, lentigos, ephiledes and seborrheic keratoses. This encompasses irradiating the skin with light (electromagnetic radiation) having a wavelength sufficient to irradiate surface pigmentation while reducing targeting of dermal blood vessels. Using a dye laser to administer wavelengths outside of the absorption peaks for hemoglobin typically used to treat unwanted blood vessels would enable removal of epidermal and dermal pigment without causing purpura in the skin.

Pulsed-dye lasers typically use wavelengths of 585 or 595 nanometers to treat blood vessels. Using these wavelengths without surface cooling and with a curved lens to push the blood out of the dermal vessels using pressure has the effect of targeting brown spots. However, this approach has the limitation of requiring each spot to be treated individually, and the further limitation of producing purpura following a second treatment to the same area of skin for blood vessel reduction. This additional treatment usually incorporates some form of epidermal cooling and is administered to the entire face or entire cosmetic unit (cheek, chin, nose for example) to be treated.

By treating the entire face with a wavelength not as highly absorbed by hemoglobin outside the wavelengths absorbed by hemoglobin, 574-598 nanometers, melanin can be targeted while avoiding strong blood absorption. This has the advantage of reducing the likelihood of purpura following treatment, and enabling treatment of the entire skin surface for pigmented lesions such as ephiledes or lentigos. This has the advantage of enabling more rapid treatment of a given area, since each individual brown spot does not have to be identified and treated, and reduces the likelihood of developing purpura post-treatment.

In another embodiment, there is disclosed a method for treating unwanted vasculature and pigmentation with the same device using two or more wavelengths of electromagnetic radiation. This method typically comprises simultaneously or sequentially administering said two or more wavelengths, wherein one is for the unwanted vasculature and the other is outside of the absorption peaks for hemoglobin.

This embodiment comprises irradiating the skin with electromagnetic radiation wavelength that ranges from 550-560 nanometers, 601-690 nanometers or combinations of these ranges. As previously describes, this radiation can be applied in a pulsed, scanned, or continuous manner.

Wavelengths of light outside of the hemoglobin absorption peak can be administered with a compression lens as described above, but this should be unnecessary since removal of hemoglobin from the target area should not dramatically change the response of a pigmented lesion to wavelengths outside of the 574-598 nanometer range. Pulsed-dye lasers have been developed in the past that were capable of being tuned for emission of wavelengths ranging from 585 nanometers to 600 nanometers in 5 nanometer increments. Wavelengths longer than 595 nanometers were not often used, since they were relatively ineffective at treating vascular lesions. Current lasers do not generally offer this broad range of wavelengths.

Development of a pulsed-dye laser capable of delivering vascular-specific wavelengths in the 574-598 nanometer range, as well as wavelengths outside of that range below 574 nanometers or above 600 nanometers for the purpose of treating pigmentation in the skin would dramatically increase the utility of pulsed-dye lasers. A diffraction gradient, prism, or other means of altering the delivered wavelength emitting from a dye laser would enable the delivery of treatments to reduce unwanted blood vessels, unwanted pigmentation and induce skin remodeling. These different wavelengths could be delivered singly, sequentially, or simultaneously to achieve the desired outcome of removing unwanted pigmentation and unwanted vasculature. A dye laser could be continuous and swept over the skin to result in the effect of a pulse, or pulsed as most lasers in clinical use today are.

A laser capable of delivering at least two wavelengths, at least one specific for hemoglobin, and at least one targeting melanin pigment and not at the peak absorption range for hemoglobin, would enable more complete treatment of sun-damaged skin and other conditions. This system would have significant advantages over intense pulsed light systems because there would be narrow ranges of wavelengths being administered as opposed to the broad wavelength ranges delivered with intense pulsed light devices. The administration of more discrete wavelengths permits more accurate prediction of laser effects than is possible when using broad spectrum light sources such as intense pulsed light devices. Intense pulsed light devices emit a broad range of wavelengths, making prediction of clinical outcomes more difficult. Using laser energy of specific wavelengths permits a more accurate estimation of the proper energy to be used for a given patient, enhancing effectiveness and limiting side effects.

Pulsed-dye lasers are currently used mostly for treating vascular lesions such as port-wine stain birthmarks, rosacea, facial veins and diffuse redness, scars and lower extremity spider veins. Recently, an attachment that allows compression of the skin to blanch redness was developed enabling better treatment of pigmented lesions. The problem with using this compression hand-piece is that it requires each lesion to be treated individually, and that a full rejuvenating treatment before or after treatment of pigmented lesions often results in significant purpura.

Pulsed-dye lasers are capable of emitting a variety of wavelengths depending upon the type of dye used in the laser through the use of diffraction gradients or prisms within the laser. Thus lasers can be tuned to different wavelengths. Incorporating the ability to tune to wavelengths not in the peak absorption range of hemoglobin absorption would enable the treatment of pigmented lesions. Melanin pigment absorbs light over a much broader range than hemoglobin. Ideal choices for melanin treatment would be from approximately 550-560 nanometers or 605-620 nanometers. The ideal wavelength would depend upon how much energy could be delivered at the various wavelengths.

Using a single dye in the dye laser to achieve both vascular-specific wavelengths and those outside the range of hemoglobin for treating pigmented lesions would simplify the laser and lower cost. Thus wavelengths up to approximately 615 nm would be most easily achievable while also delivering vascular-specific wavelengths such as 595 nm. Combining the ability to deliver a wavelength of light not in the peak absorption range of hemoglobin would enable treatment of epidermal and dermal melanocytic lesions without affecting dermal blood vessels and causing a bruise. The wavelength specific for hemoglobin, most commonly 595 nanometers in current clinical practice, could be administered before, during or concurrently with the 595 nanometer wavelength. This would enable treatment of melanocytic lesions such as ephiledes or lentigos with a lower risk of purpura.

The present disclosure is further illustrated by the following non-limiting examples, which is intended to be purely exemplary of the disclosure.

### EXAMPLES

### Example 1

The following treatments were administered to the arm of a patient using a pulsed-dye laser comprising a rhodamine dye. Electromagnetic radiation (light) was administered to three separate areas of the arm of the patient containing multiple freckles. In each of the areas, electromagnetic radiation having a wavelength of 607, a 10 mm spot size, a pulse duration of 1.5 milliseconds was administered. The three separate areas were treated with an average fluence of 3.0, 4.0 and 5.0 Joules per square centimeter, respectively.

All three treatment areas initially showed minimal pinkness of the skin, with no purpura. After 24 hours the treated spots turned dark, showing increased pigmentation. Within two weeks of the initial treatment the darker spots were gone and the skin returned to a normal appearance.

### Example 2

Like Example 1, the following treatment was administered to the arm of a patient using a pulsed-dye laser comprising a rhodamine dye. Electromagnetic radiation (light) was administered to an area of the arm containing multiple freckles. In the area, electromagnetic radiation having a wavelength of 607, a 7.0 mm spot size, a pulse duration of 1.5 milliseconds, and an average fluence of 10.0 Joules per square centimeter.

As in Example 1, the treated area initially showed minimal pinkness of skin, with no purpura. After 24 hours the treated spots turned dark, showing increased pigmentation. Within two weeks of the initial treatment the darker spots were gone and the skin returned to a normal appearance.

As indicated by these results, a method of treating the skin with laser light according to present disclosure shows significant improvement in removing brown spots, such as freckles from the skin.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating pigmented lesions on the skin,
said method comprising irradiating the skin with electromagnetic radiation having a wavelength that is absorbed more efficiently by the pigmented lesion than by dermal blood vessels or hemoglobin.

2. The method of claim 1, wherein said electromagnetic radiation comprises laser light.

3. The method of claim 1, wherein said wavelength ranges from 550-560 nanometers, 601-690 nanometers or combinations of these ranges.

4. The method of claim 1, wherein said electromagnetic radiation is applied in a pulsed, scanned, or continuous manner.

5. The method of claim 1, further comprising irradiating the skin with electromagnetic radiation having a wavelength ranging from 570-598 nanometers for removing vascular lesions and/or dermal remodeling.

6. The method of claim 5, wherein said further irradiating occurs prior to, subsequent to or simultaneous with irradiating the skin with electromagnetic radiation having a wavelength that is absorbed more efficiently by the pigmented lesion.

7. The method of claim 6, wherein said method comprises sequentially administering at least two wavelengths of electromagnetic radiation, one for unwanted vasculature and one outside of the absorption peaks for hemoglobin for treating pigmentation.

8. The method of claim 6, wherein said method comprises simultaneously administering at least two wavelengths of electromagnetic radiation, one for unwanted vasculature and one outside of the absorption peaks for hemoglobin for treating pigmentation where the two or more wavelengths are administered.

9. The method of claim 1, wherein said electromagnetic radiation is administered using a dye laser.

10. The method of claim 9, further comprising treating the skin prior to or after said irradiation step with topical agents chosen from alpha-hydroxy acids, retinoids, or antioxidants for the purpose of rejuvenating aged or photodamaged skin.

11. The method of claim 9, further comprising treating the skin prior to or after said irradiation step with microdermabrasion or chemical peels for rejuvenating aged or photodamaged skin.

12. The method of claim 9, further comprising treating the skin prior to or after said irradiation step with other electromagnetic radiation chosen from radiofrequency, fractional laser, intense pulsed-light using islands of sparing, infrared pulsed, scanned, or continuous radiation, low-level electromagnetic radiation administered using light emitting diodes (LEDs), and combinations thereof.

13. A method of treating unwanted vasculature and pigmentation of the skin with the same device using two or more wavelengths of electromagnetic radiation, said method comprising simultaneously or sequentially administering said two or more wavelengths, wherein one wavelength is for said unwanted vasculature and the other is outside of the absorption peaks for hemoglobin,
said method comprising irradiating the skin with electromagnetic radiation having a wavelength 550-560 nanometers, 601-690 nanometers or combinations of these wavelength ranges.

14. The method of claim 13, wherein said electromagnetic radiation comprises laser light that is applied in a pulsed, scanned, or continuous manner.

15. The method of claim 13, further comprising irradiating the skin with electromagnetic radiation having a wavelength ranging from 570-598 nanometers for removing vascular lesions and/or dermal remodeling.

16. The method of claim 15, wherein said further irradiating occurs prior to, subsequent to or simultaneous with irradiating the skin with electromagnetic radiation having a wavelength that is absorbed more efficiently by the pigmented lesion.

17. The method of claim 13, wherein said electromagnetic radiation is administered using a dye laser.

18. The method of claim 17, wherein said dye comprises rhodamine.

19. The method of claim 13, further comprising treating the skin prior to or after said irradiation step with topical agents chosen from alpha-hydroxy acids, retinoids, or antioxidants for the purpose of rejuvenating aged or photodamaged skin.

20. The method of claim 19, further comprising treating the skin prior to or after said irradiation step with microdermabrasion or chemical peels for rejuvenating aged or photodamaged skin.

21. The method of claim 19, further comprising treating the skin prior to or after said irradiation step with other electromagnetic radiation chosen from radiofrequency, fractional laser, intense pulsed-light using islands of sparing, infrared pulsed, scanned, or continuous radiation, low-level electromagnetic radiation administered using light emitting diodes (LEDs), and combinations thereof.
